(19) United States
(12) Patent Application Publication
(10) Pub. No.: US 2025/0332561 A1
(43) Pub. Date: Oct. 30, 2025

(54) APPARATUS FOR SYNTHESIZING NUCLEIC ACID AND NUCLEIC ACID SYNTHESIS METHOD USING THE SAME

(71) Applicants: Samsung Electronics Co., Ltd., Suwon-si (KR); Korea University Research and Business Foundation, Seoul (KR)

(72) Inventors: Sunghaeng Lee, Suwon-si (KR); Honggu Chun, Seoul (KR); Nagjong Kim, Suwon-si (KR)

(21) Appl. No.: 18/908,305

(22) Filed: Oct. 7, 2024

(30) Foreign Application Priority Data
Apr. 25, 2024 (KR) 10-2024-0055580

(51) Int. Cl. B01J 19/00

(52) U.S. Cl. CPC B01J 19/0046; B01J 2219/00454; B01J 2219/00533; B01J 2219/00713; B01J 2219/00722

(57) ABSTRACT

An apparatus for synthesizing nucleic acids and a method for synthesizing nucleic acids using the same. The apparatus includes a first substrate including a surface on which the nucleic acids are synthesized, a second substrate including a deprotection solution, a transporter configured to move the first substrate or the second substrate, and a transporter controller in operable communication with the transporter.

### CROSS-REFERENCE TO RELATED APPLICATION

This application claims priority to Korean Patent Application No. 10-2024-0055580, filed on Apr. 25, 2024, in the Korean Intellectual Property Office, and all benefits accruing therefrom under 35 USC § 119, the entire content of which is incorporated by reference herein.

### BACKGROUND

### 1. Field

The disclosure relates to an apparatus for synthesizing nucleic acids and a method for synthesizing nucleic acids by using the same.

### 2. Description of the Related Art

Nucleic acid synthesis may be performed by solid-phase chemical synthesis. Solid-phase chemical synthesis of nucleic acids includes phosphoramidite synthesis methods. This method relies heavily on phosphoramidite chemistry, such as solid-phase column-based synthesis, and microarray-based photoactivated synthesis. However, these solid-phase synthesis methods have significant error rates, and have limitations in synthesizing practical lengths of nucleic acids.

Therefore, there is a need for an apparatus for synthesizing nucleic acids, which can efficiently synthesize nucleic acids with existing technologies, and a method for synthesizing nucleic acids using the same.

### SUMMARY

According to an aspect, an apparatus for synthesizing nucleic acids, includes a first substrate including a surface on which the nucleic acids are synthesized, the surface including a nucleic acid synthesis region functionalized to immobilize the nucleic acids or nucleic acid precursors; a second substrate including a deprotection solution, wherein the second substrate includes an array of regions capable of including the deprotection solution, an array of first electrodes in operable communication with the regions and configured to induce electrochemical acid generation in the deprotection solution, and a first electrode controller in operable communication with the array of the first electrodes to apply a voltage to the first electrodes and to remove the voltage from the first electrodes; a transporter configured to move the first substrate or the second substrate, wherein the transporter is in operable communication with the first substrate or the second substrate, and the transporter is configured to align the first substrate and the second substrate with each other; and a transporter controller in operable communication with the transporter. The transporter controller is configured to i) direct the transporter to move the first substrate in a direction toward or away from the second substrate or ii) direct the transporter to move the second substrate in a direction toward or away from the first substrate.

According to another aspect, a method for synthesizing nucleic acids, includes identifying a target nucleic acid sequence to be synthesized; providing a first substrate having a functionalized surface to immobilize the nucleic acids or nucleic acid precursors, aligning the first substrate with a second substrate in operable communication with an array of first electrodes, wherein the second substrate includes a deprotection solution; applying a voltage to the array of first electrodes to induce an acid generation for deprotection; and aligning the first substrate with a first bulk reagent substrate, wherein the first bulk reagent substrate includes a coupling reagent.

Additional aspects will be set forth in part in the detailed description which follows and, in part, will be apparent from the detailed description, or may be learned by practice of certain exemplary embodiments.

### BRIEF DESCRIPTION OF THE DRAWINGS

The above and other aspects, features, and advantages of certain exemplary embodiments will be more apparent from the following detailed description taken in conjunction with the accompanying drawings, in which:

FIG. 1 is a diagram schematically illustrating an embodiment of a process of nucleic acid synthesis by a phosphoramidite method;

FIG. 2 is a diagram schematically illustrating an embodiment of a nucleic acid synthesis apparatus and a nucleic acid synthesis process using the same;

FIGS. 3A, 3B, and 3C are diagrams schematically illustrating an embodiment of a nucleic acid synthesis apparatus and a nucleic acid synthesis process using the same;

FIGS. 4A and 4B are diagrams illustrating an embodiment of a nucleic acid synthesis apparatus, and electrode structures of a first substrate and a second substrate;

FIG. 5 is a diagram illustrating an embodiment of a nucleic acid synthesis apparatus, and electrode structures of a first substrate and a second substrate;

FIG. 6 is a diagram illustrating an embodiment of a nucleic acid synthesis apparatus, and electrode structures of a first substrate and a second substrate;

FIG. 7 is a diagram illustrating an embodiment of a nucleic acid synthesis apparatus including electrodes connected to a nucleic acid synthesis region of a first substrate;

FIG. 8 is a diagram illustrating an embodiment of a nucleic acid synthesis apparatus including electrodes connected to a nucleic acid synthesis region of a first substrate;

FIG. 9 is a diagram illustrating an embodiment of a nucleic acid synthesis apparatus including electrodes connected to a nucleic acid synthesis region of a first substrate;

FIG. 10 is a diagram illustrating an embodiment of a nucleic acid synthesis apparatus including electrodes connected to a nucleic acid synthesis region of a first substrate;

FIG. 11 is a diagram illustrating an embodiment of a nucleic acid synthesis apparatus not including electrodes connected to a nucleic acid synthesis region of a first substrate;

FIG. 12 is a diagram illustrating an embodiment of a nucleic acid synthesis apparatus not including electrodes connected to a nucleic acid synthesis region of a first substrate;

FIG. 13 is a diagram illustrating an embodiment of a nucleic acid synthesis apparatus not including electrodes connected to a nucleic acid synthesis region of a first substrate;

FIG. 14 is a diagram illustrating an embodiment of a nucleic acid synthesis apparatus not including electrodes connected to a nucleic acid synthesis region of a first substrate; and

FIGS. 15A and 15B are schematic diagrams illustrating an electric field in relation to the electrode structure of an embodiment of a nucleic acid synthesis apparatus.

### DETAILED DESCRIPTION

Reference will now be made in detail to exemplary embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain certain aspects. As used herein, the term “and/or” includes any and all combinations of one or more of the associated listed items. Expressions such as “at least one of,” when preceding a list of elements, modify the entire list of elements and do not modify the individual elements of the list.

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

According to an aspect, the apparatus for synthesizing nucleic acids includes a first substrate, a second substrate, a transporter, and a transporter controller. The first substrate includes a surface on which the nucleic acids are synthesized. The surface includes a nucleic acid synthesis region functionalized to immobilize the nucleic acids or nucleic acid precursors. The second substrate includes a deprotection solution an array of regions capable of including the deprotection solution, an array of first electrodes in operable communication with the regions and configured to induce electrochemical acid production in the deprotection solution, and a first electrode controller in operable communication with the array of the first electrodes and configured to apply a voltage to the first electrodes and to remove the voltage from the first electrodes. The transporter is configured to move the first substrate or the second substrate, wherein the transporter is in operable communication with the first substrate or the second substrate, and the transporter is configured to align the first substrate and the second substrate with each other. The transporter controller is in operable communication with the transporter. The transporter controller is configured to i) direct the transporter to move the first substrate in a direction toward or away from the second substrate or ii) direct the transporter to move the second substrate in a direction toward or away from the first substrate.

As used herein, the term “nucleic acid” refers to a polymer in which two or more nucleotides are linked. The nucleic acid is also called a polynucleotide. The nucleic acid may be deoxyribonucleic acid (DNA) or ribonucleic acid (RNA). The nucleic acid may be a single-stranded nucleic acid or a double-stranded nucleic acid. The nucleic acid may include a natural nucleotide as well as a modified nucleotide.

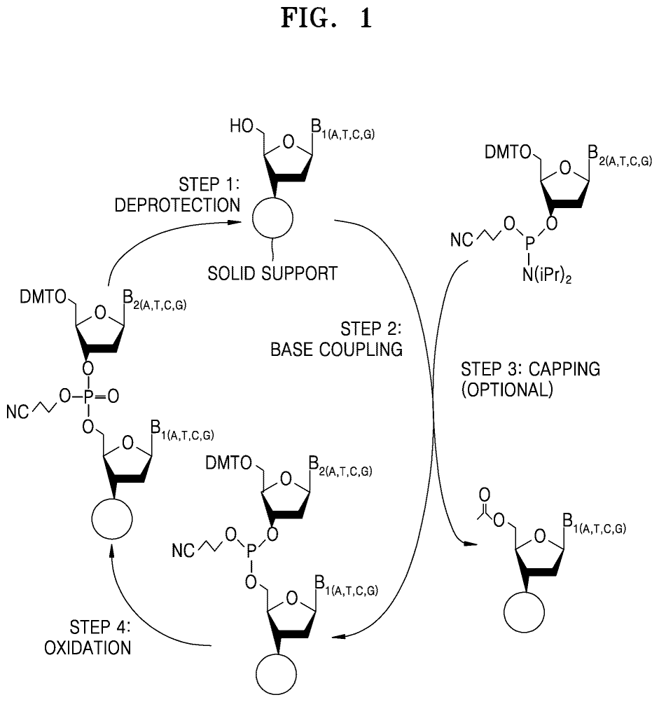

As used herein, the nucleic acid synthesis may be a solid-phase chemical synthesis. The solid-phase chemical synthesis may be performed using a phosphoramidite synthesis method. FIG. 1 provides an example of nucleic acid synthesis by phosphoramidite synthesis method. In FIG. 1, dimethoxytrityl (DMT) is a 5′-OH protecting group, and B represents the base that may be protected. The base may be an adenine (A) base, a thymine (T) base, a guanine (G) base, or a cytosine (C) base. Adenine, guanine, or cytosine bases may have a protecting group (primary amine protecting group) on a primary amino functional group to protect the primary amino functional group from side reactions during the coupling. The primary amine protecting group may be removed at the conclusion of the nucleic acid synthesis. The thymine base may be provided without a protecting group on a functional group (e.g., the thymine base may be unprotected). In the method, protected and linkable variants of nucleosides are linked together in a chain through a cycle of activation and coupling (e.g., repeated cycles of activation and coupling). As shown in FIG. 1, the synthesis cycle may be as follows: generally, the synthesis begins from a solid support, such as a glass surface, and the starting nucleotides are attached to the solid support via a cleavable linker. During this step, or the cycle in general, the previous base is in place as a nucleoside with a trityl protecting group (DMT). Step 1 of the cycle is to remove the trityl protecting group, i.e., the “detritylation” or “deprotection” step, exposing the reactive 5′-OH group of the sugar ring. The deprotection may be performed by introduction of an acid in a suitable solvent, such as trichloroacetic acid (TCA) in the solvent dichloromethane. The introduction of the acid may be accomplished by electrochemical methods. Step 2 is to introduce the next phosphoramidite with the desired next base for the coupling reaction. The introduced phosphoramidite may be provided in the solvent acetonitrile, and its diisopropylamino group may be activated or protonated by mixing in an acidic catalyst such as ETT (5-(ethylthio)-1H-tetrazole) to facilitate the coupling reaction. The product may be one that forms a phosphite triester linkage in the nucleoside chain and a free diisopropylamino group (iPr).

Step 3 is an optional step (“capping step”), and may be used to protect unreacted 5′-OH in the coupling reaction to prevent further chain extension. The unreacted 5′-OH may be, for example, acetylated as shown in FIG. 1. Use of a capping step may reduce or prevent single base deletions which lead to reduced product yield.

Step 4 is an oxidation step to convert the phosphite group to the more stable phosphate form. The oxidation of the phosphite triester may be achieved by adding iodine and pyridine in water. The product is a phosphate triester, a standard nucleic acid backbone with the cyanoethyl (CN) protecting group remaining on the free oxygen. The cycle (e.g., a synthesis cycle) of deprotection, coupling, optionally capping, and oxidation may be repeated until the desired strand sequence of bases and chain length is provided by the process. Upon completion of the desired number of cycles, the synthesis product may be released from the solid support by a cleavage reaction.

In the nucleic acid synthesis apparatus, the first substrate may include electrodes corresponding to the electrodes of the second substrate. The term “corresponding” may refer to being installed on each or all of the electrodes of the second substrate in a direction facing each other. The electrodes connected to the first substrate may be a large-area electrode. The large-area electrode may be partially or fully connected to the surface of the first substrate. The electrodes may be flat electrodes.

In the first substrate, the nucleic acid synthesis region may be a micro-area region where a nucleic acid of a specific sequence is synthesized. The region may be a surface region. The region may be arranged in a predetermined pattern. The region may be arranged according to the sequence of the target nucleic acid to be synthesized. The region may be in the form of a flat surface or a well. The region may be an array of flat surfaces, an array of wells, or a combination thereof. The region may be in contact with the deprotection solution included in the second substrate. The nucleic acid synthesis region may have a diameter of 35 micrometers (μm) or less, for example, about 0.1 μm to about 12 μm, about 0.1 μm to about 10 μm, about 0.1 μm to about 5 μm, or about 0.1 μm to about 1 μm.

The region may be functionalized to immobilize phosphoramidite and support the nucleic acid synthesis through the addition of introduced phosphoramidite. The nucleic acid or the nucleic acid precursor may be linked to a chemical functionalization layer, such as silane disposed on (e.g., attached or chemically bonded) the region. Additionally, the nucleic acid or the nucleic acid precursor may be attached to the region through a suitable chemical bond or linker group. The nucleic acid or the nucleic acid precursor may be a protected nucleoside or a single-stranded nucleic acid. The protected nucleoside may be protected with a known protecting group, for example, 4,4′-dimethoxytrityl (DMT).

With the first substrate and the second substrate aligned, the growing nucleoside chain in the region may be deprotected by H⁺ ions generated at the first electrodes of the second substrate. The deprotected group may be located within about 1 nanometer (nm), within about 10 nm, within about 20 nm, within about 50 nm, or within about 100 nm of the surface of the first electrodes. As used herein, the electrodes may be made of a material consisting of gold (Au), aluminum (Al), titanium (Ti), copper (Cu), platinum (Pt), or a combination thereof. As used herein, an “electrode” may be a microelectrode whose horizontal and vertical lengths are each less than a micrometer. The electrodes may have a horizontal and vertical length of less than 100 μm, less than 50 μm, less than 10 μm, less than 1 μm, less than 0.5 μm, or less than 0.1 μm, respectively.

In the second substrate, the “deprotection solution” may be a solution that may be applied to deprotect (i.e., remove) a protecting group. The protecting group may be a protecting group used in chemical synthesis of nucleic acids, such as DMT. The DMT is also called “trityl” and is a group protected at the 5′ hydroxyl position of a nucleoside. The protected nucleic acid may be a protected phosphoramidite.

The deprotection solution may be a compound that generates acid through electrochemical redox. The electrochemical redox may be provided by application of a voltage to the first electrodes. The deprotection solution may include a reversible redox pair. The reversible redox pair may include hydroquinone (HQ), and benzoquinone (BQ), such as tetra-1,4-benzoquinone (TQ). The redox reaction may occur in aqueous and non-aqueous solvents.

In the second substrate, in the array of regions capable of including the deprotection solution, the regions may have a form of a flat surface or a well. The flat surface region may be a region where H⁺ ions are limited by one or more first electrodes. As used herein, the region may have a diameter of 35 μm or less, for example, about 0.1 μm to about 12 μm, about 0.1 μm to about 10 μm, about 0.1 μm to about 5 μm, or about 0.1 μm to about 1 μm.

In the second substrate, the region that may include the deprotection solution of the second substrate may include a well. The well may be an array of wells. The array of electrodes may be operably connected to each well of the array of wells. In the case of alignment between the first substrate and the second substrate, the surface on which the nucleic acid of the first substrate is synthesized and a part of the well may be in contact (e.g., a top of the well), allowing the growing nucleoside chain immobilized on the surface to contact the deprotection solution in the well.

In the second substrate, each electrode of the array of electrodes may include two electrodes or three electrodes. The two electrodes may be a working electrode and a standard electrode. The three electrodes may be a working electrode, a counter electrode, and a standard electrode. Each electrode of the array of electrodes may include an insulating layer between electrodes, one electrode may generate H⁺ ions, and another electrode may generate OH-ions. The generated H⁺ ions may be limited by the insulating layer and the generated OH-ions without moving to other areas. The first electrode may be a flat electrode. Each electrode of the array of electrodes may have an address-specific voltage applied thereto.

In the second substrate, the first electrode controller may be in operable communication with the first electrodes and may be able to individually access and control each electrode. The controller may be a switch that applies or blocks voltage to the first electrodes. The controller may be a metal-oxide-semiconductor field-effect transistor (MOSFET), such as a complementary MOSFET (CMOS), an insulated-gate bipolar transistor (IGBT), or a bipolar junction transistor (BJT).

The nucleic acid synthesis apparatus may include a control processor in operable communication with the first electrode controller.

In the nucleic acid synthesis apparatus, the transporter may be in operable communication with the first substrate or the second substrate, and may move the first substrate or the second substrate, respectively. The movement may be moving the surface on which the nucleic acid of the first substrate is synthesized toward or away from the region including the deprotection solution of the second substrate. The transporter may align the first substrate and the second substrate by moving the first substrate, the second substrate, or the first substrate and the second substrate. By the alignment, the region including the deprotection solution between the first substrate and the second substrate may be a closed region. The closed region may be in the form of a chamber. The transporter may include a driving means. The driving means may be a motor, for example, a micromotor.

In the nucleic acid synthesis apparatus, a transporter controller may be in operable communication with the transporter, and may direct the transporter to move the first substrate or the second substrate in a direction toward or away from the second substrate or the first substrate. The movement may be used to align the first substrate and the second substrate. By the alignment, the region including the deprotection solution between the first substrate and the second substrate may be closed. The closed region may be in the form of a chamber. By the movement, the growing nucleoside chain of the first substrate may come into contact with the deprotection solution of the second substrate, thereby deprotecting the protected nucleoside chain in the selected region.

The apparatus for synthesizing a nucleic acid may further include an alignment sensor and an alignment controller. The alignment sensor may be functionally connected between (e.g., in operable communication with) the first substrate and the second substrate, and may be configured to measure horizontal plane misalignment between the first substrate and the second substrate.

The alignment controller may be operable communication with the alignment sensor and configured to receive a horizontal plane misalignment from the alignment sensor, and to compare the horizontal plane misalignment with a misalignment threshold. When the misalignment is greater than the misalignment threshold, the alignment controller may instruct the transporter controller to move the transporter in a direction in which the horizontal plane misalignment decreases. When the horizontal plane misalignment is less than or equal to the misalignment threshold, the alignment controller may send an alignment acknowledgment to the transporter controller. The threshold may be set to 150 nm or less, for example, 100 nm or less.

The nucleic acid synthesis apparatus may further include a bulk reagent substrate including a bulk reagent. The bulk reagent substrate may have a container shape capable of including a bulk reagent. The bulk reagent substrate may be in a form having a container, for example, only one container capable of including a bulk reagent. The bulk reagent substrate may include a bulk reagent, and the bulk reagent may be disposed on (e.g., in contact with, or in direct contact with) the entire synthesis area of the first substrate. The bulk reagent substrate may not include an array of wells. The bulk reagent substrate may not include an array of electrodes. The bulk reagent substrate may include a bulk liquid reagent. The bulk liquid reagent may be a coupling reagent, a capping reagent, or an oxidizing reagent.

The nucleic acid synthesis apparatus may further include a second transporter in operable communication with the first substrate and configured to advance the first substrate toward the bulk reagent substrate or retreat from the bulk reagent substrate. The bulk reagent substrate may include a plurality of bulk reagent substrates (e.g., a first bulk reagent, a second bulk reagent substrate, and a third bulk reagent substrate), and each bulk reagent substrate may include a coupling reagent, a capping reagent, or an oxidation reagent.

Additionally, the nucleic acid synthesis apparatus may include a fluid supply unit that supplies fluid to the first substrate, the second substrate, the bulk reagent substrate, or a combination thereof. The fluid supply unit may supply a liquid reagent to each of the first substrate, the second substrate, and/or the bulk reagent substrate.

Additionally, the nucleic acid synthesis apparatus may include a washing unit for washing the first substrate, the second substrate, the bulk reagent substrate, or a combination thereof. The washing unit may wash the nucleic acid synthesis region of the first substrate, the region including a deprotection reagent of the second substrate, and the region including the bulk reagent of the bulk reagent substrate. The washing unit may supply fluid to the first substrate, the second substrate, the bulk reagent substrate, or a combination thereof in order to wash it/them. The fluid may be liquid. The liquid may be water or a buffer. The washing unit may remove reagents from each substrate.

According to another aspect, the method for synthesizing a nucleic acid includes identifying a target nucleic acid sequence to be synthesized, providing a first substrate having a functionalized surface to immobilize the nucleic acid or a nucleic acid precursor, aligning the first substrate with a second substrate in operable communication with an array of first electrodes wherein the second substrate includes a deprotection solution, applying a voltage to the array of first electrodes to induce acid generation for deprotection, and aligning the first substrate with a first bulk reagent substrate, wherein the first bulk reagent substrate includes a coupling reagent.

In identifying a target nucleic acid sequence to be synthesized, the nucleic acid may be DNA or RNA.

In providing a first substrate having a surface functionalized to immobilize the nucleic acid or the nucleic acid precursor thereof, the first substrate is the same as described herein

In aligning the first substrate with the second substrate, the second substrate is the same as described herein. The alignment may be such that the nucleic acid synthesis region of the first substrate is brought into contact (e.g., direct contact) with the deprotection solution of the second substrate. The alignment may be performed using an alignment sensor and an alignment controller. The approach may be such that a sealed well or chamber (e.g., a closed well, a closed chamber, or the like) is created.

In applying a voltage to the array of first electrodes on the second substrate to induce acid generation for deprotection, the second substrate may be an acid generating compound when the voltage is applied. The compound may include a reversible redox pair. The solution may include hydroquinone, benzoquinone, or tetra-1,4-benzoquinone. The first bulk reagent substrate includes a coupling reagent. The coupling reagent may be a reagent including a nucleic acid monomer such as DNA or RNA having a specific base according to the target nucleic acid sequence identified in “identifying a target nucleic acid sequence to be synthesized”. Applying the voltage may include address-specifically applying the voltage to a specific electrode of the array of first electrodes. The array of first electrodes may be a CMOS, a IGBT, or a BJT.

The method may further include after aligning the first substrate with the first bulk reagent substrate, aligning the first substrate with a second bulk reagent substrate including a capping reagent, aligning the first substrate with a third bulk reagent substrate including an oxidizing reagent, or aligning the first substrate with the second bulk reagent substrate including the capping reagent and aligning the first substrate with the third bulk reagent substrate including the oxidizing reagent.

Each of the above synthesis steps may be repeated using different types of protected bases. The method may be performed using the nucleic acid synthesis apparatus as described herein.

The nucleic acid synthesis apparatus according to an aspect may be used to efficiently synthesize nucleic acids.

By a method for synthesizing a nucleic acid according to an aspect, nucleic acids may be efficiently synthesized. In the above method, the second substrate on which the deprotection is performed may be reused, saving costs and reagent quantities, thereby increasing productivity and process efficiency.

Hereinafter, the disclosure will be described in more detail through exemplary embodiments. However, the present embodiments are for illustrative purposes only and the scope of the embodiments is not limited to these exemplary embodiments.

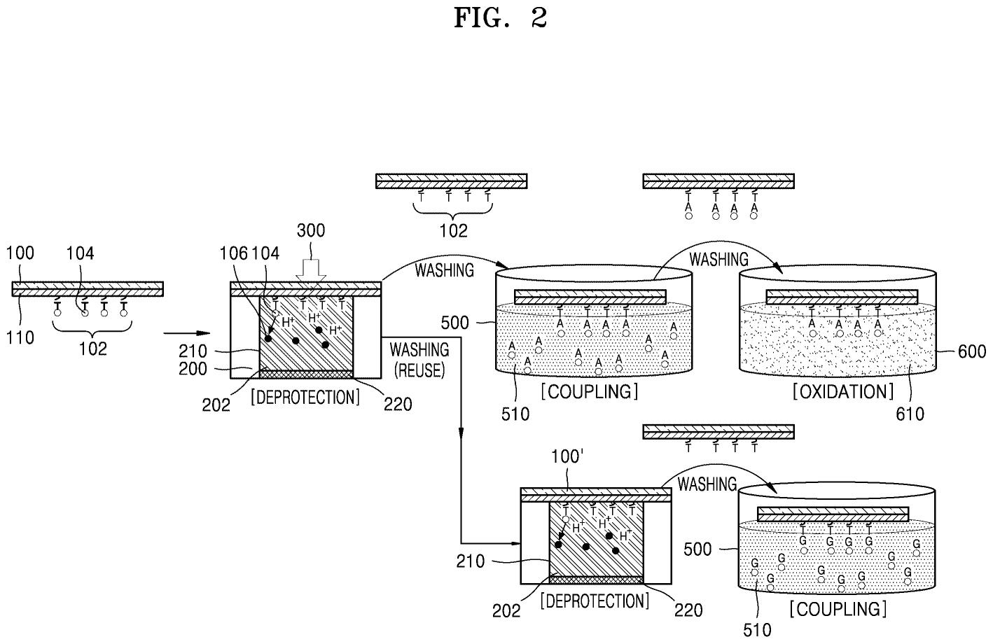

FIG. 2 is a diagram schematically illustrating an embodiment of a nucleic acid synthesis apparatus and a nucleic acid synthesis process using the same. In FIG. 2, for convenience of explanation, only one well and one electrode are shown on the second substrate. The second substrate may further comprise a plurality of wells and/or a plurality of electrodes.

As shown in FIG. 2, a first substrate 100 is disposed on an electrode 110 and includes a nucleic acid synthesis region 102 on a surface of the electrode 110. In the nucleic acid synthesis region 102, a nucleic acid or a nucleic acid precursor protected with a protecting group 104 may be attached. A second substrate 200 includes a well 210 including a deprotection reagent 202 and a first electrode 220 disposed on the bottom of the well.

The first substrate 100 may be aligned with the second substrate 200 including the deprotection reagent 202 by a transporter 300, and a voltage may be applied to the first electrode 220 of the second substrate toward the electrode 110 of the first substrate, the protecting group 104 may be deprotected by acid generated from the applied voltage to provide a released protecting group 106. Accordingly, the well 210 of the second substrate includes the protecting group 106 released from the protected nucleic acid or nucleic acid precursor. At this time, the electrode 220 of the second substrate may be a reference electrode and the electrode 110 of the first substrate may be a working electrode. The alignment may be controlled by an alignment sensor (not shown).

After the deprotection reaction is completed, the nucleic acid synthesis region 102 of the first substrate may be selectively washed and then moved by the transporter and aligned with a first bulk reagent substrate 500. The first bulk reagent substrate 500 may be in the form of a well or a chamber including a coupling reagent 510. The first bulk reagent substrate may include protected nucleotides with the bases to be added in the nucleic acid sequence. The protected nucleotide may be protected versions of A, T, G, or C bases. The aligned first substrate and first bulk reagent substrate may be incubated in an aligned state to allow a coupling reaction to occur.

Optionally, after completion of the coupling reaction, the first substrate may be moved by the transporter to align with a second bulk reagent substrate (not shown). The second bulk reagent substrate may include a capping reagent. The second bulk reagent substrate may be in the form of a well or a chamber including a capping reagent. The capping reagent may include a group that protects unreacted 5′-OH in the synthesized chain. For example, the capping reagent may include acetic anhydride and N-methylimidazole. The capping may be acetylation of 5′-OH. The aligned first substrate and second bulk reagent substrate may be incubated to allow a capping reaction to occur. With a capping step, the capped unreacted 5′-OH may be combined with the protected nucleotide in the next coupling reaction without adding nucleotides. Without a capping step, a strand including the unreacted 5′-OH group may be synthesized into a nucleic acid including one nucleotide deletion. Therefore, the capping step is optional and may be used to prevent the one nucleotide deletion from occurring. In one or more embodiments, after completion of the coupling reaction, the first substrate may not be aligned with the second bulk reagent substrate including the capping reagent, but may be directly aligned with a third bulk reagent substrate including an oxidizing reagent.

After the coupling reaction or capping reaction is completed, the first substrate may be moved by the transporter and aligned with a third bulk reagent substrate 600. The third bulk reagent substrate may include an oxidizing reagent 610. The third bulk reagent substrate may be in the form of a well or a chamber including the oxidizing reagent. The oxidizing reagent may be a reagent that oxidizes a phosphite group to a phosphate group. The oxidizing reagent may include iodine. After the coupling reaction or capping reaction is completed, the nucleic acid synthesis region of the first substrate may be optionally washed. The aligned first substrate and third bulk reagent substrate may be incubated to allow an oxidation reaction to occur.

Upon completion of the oxidation reaction, the cycle in which one nucleotide is attached to the growing strand is completed. The deprotection, coupling, optionally capping, and oxidation reactions may then be repeated using next protected nucleotides, depending on the sequence of the target nucleic acid.

After the oxidation reaction is completed, the first substrate may be moved by the transporter and aligned with the second substrate. The second substrate may include a deprotection solution in advance or a deprotection solution may be added after alignment of the first substrate and the second substrate. The second substrate may include the deprotection solution in advance in entire regions capable of including the deprotection solution. The entirety of a region may be the entirety of an array of regions. The deprotection solution may be one used in the deprotection reaction of the previous cycle. The second substrate may be one including the deprotection solution used in the previous cycle. The second substrate may be aligned not only with a first substrate including the growing strand where one nucleotide is extended in the previous cycle, but also with a different first substrate 100′, for example, with a different first substrate 100′ including the first growing strand. Accordingly, the nucleic acid synthesis apparatus may include two or more of first substrates, and each first substrate may be aligned with a second substrate including the same deprotection solution. The first substrate and the second substrate aligned may be incubated to cause a deprotection reaction. The deprotection reaction is as described above. After the deprotection reaction, the first substrate may be aligned with a bulk reagent substrate including the next sequence of protected nucleotides and incubated, as described above, to allow the coupling reaction to occur. Accordingly, the second substrate may be reused.

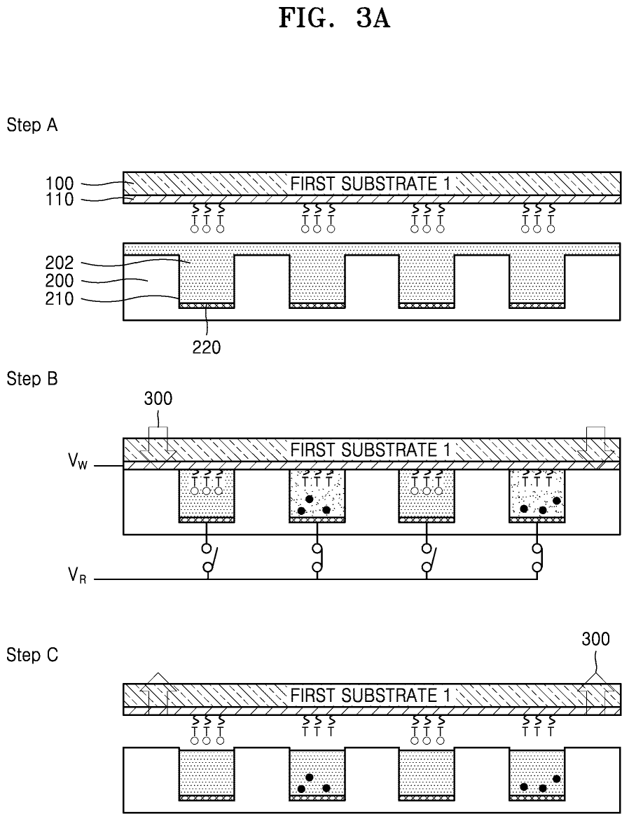

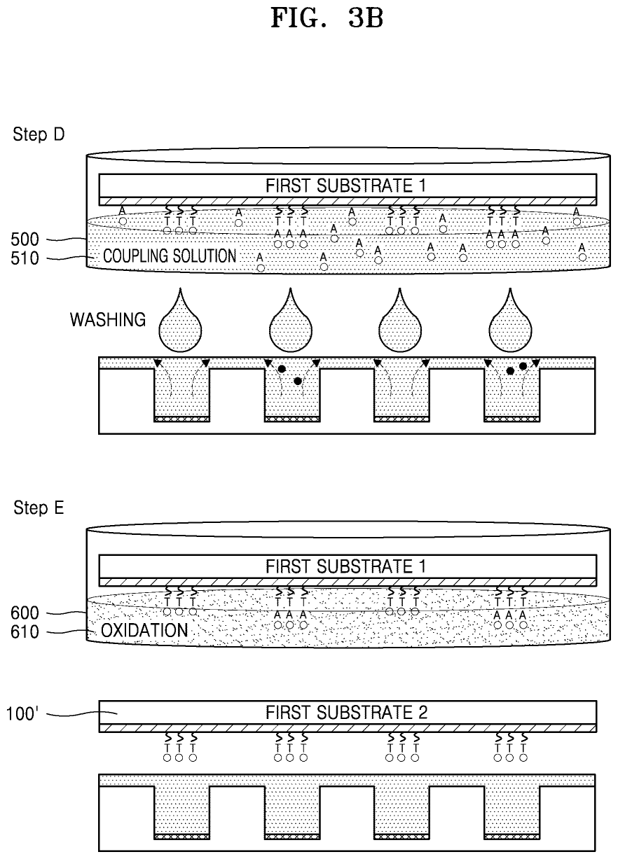

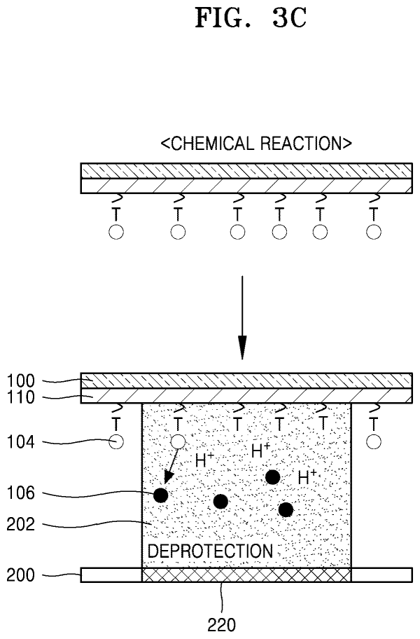

FIGS. 3A and 3B are diagrams schematically illustrating another embodiment of a nucleic acid synthesis apparatus and a nucleic acid synthesis process using the same. In FIGS. 3A and 3B, a four-well array is shown on the second substrate for convenience of explanation.

As shown in FIGS. 3A and 3B, the nucleic acid synthesis apparatus includes a first substrate 100 (labeled “First Substrate 1” in FIGS. 3A and 3B) and a second substrate 200 (Step A). The first substrate and the second substrate may be aligned by a transporter 300 (Step B). The second substrate includes an array of a plurality of wells 210, each well may be disposed on a reference electrode 220, and each reference electrode may be in operable communication with a switch and a controller (V_{w}, V_{R}). The switch may be a CMOS. The controller (V_{w}, V_{R}) may provide or may control an electrical signal instructing opening and closing of the switch. In FIG. 3A, electrodes, switches, and controllers are shown only in step B for convenience of explanation. After alignment, the electrode of the well at a specific position may be position-specifically (e.g., address-specifically) activated, for example, a voltage may be applied to generate acid. The acid generation may cause a deprotection reaction to occur. The aligned first substrate and second substrate may be incubated together or separately from the electrode activation.

The aligned first substrate and second substrate may be separated by a transporter 300 (Step C).

The separated first substrate may be aligned with a bulk coupling reagent substrate 500 (Step D in FIG. 3B). The bulk coupling reagent substrate may include a coupling reagent 510. After alignment, the first substrate and the bulk coupling reagent substrate may be incubated to perform a coupling reaction. The separated second substrate may be washed to remove the coupling reagent.

After the coupling reaction is completed, the first substrate may be separated from the coupling reaction reagent substrate and aligned with an oxidation reaction reagent substrate 600 (Step E). The washed second substrate may be aligned with a different first substrate 100′ (labeled “First Substrate 2” in FIG. 3B). The different first substrate may be a first substrate including a different cycle number of the synthesis cycles of the growing strand, or an initiating protected nucleotide.

FIG. 3C is a diagram illustrating an embodiment of an electrochemical reaction in which a deprotection reaction occurs in a state that the first substrate 100 and the second substrate 200 are aligned. Protected nucleic acids or protected nucleic acid precursors are immobilized on a first substrate, such as by a chemical reaction. When a voltage is applied to the first electrode 220 of the second substrate toward the electrode 110 of the first substrate, H+ ions are generated by redox pairs in the deprotection solution 202, and the protecting group 104 may be separated by H+ ions from the protected nucleic acid to provide a released protecting group 106 and an unprotected nucleic acid.

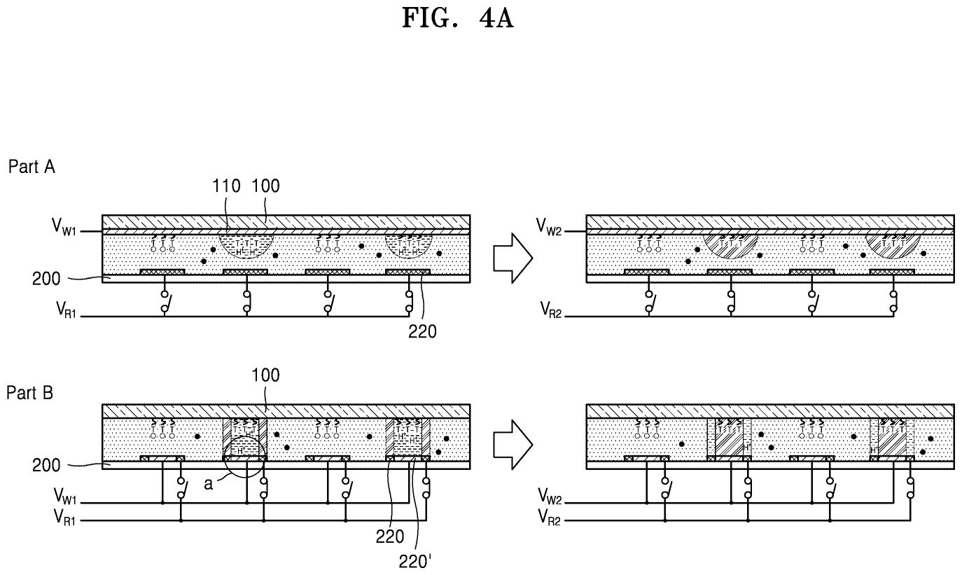

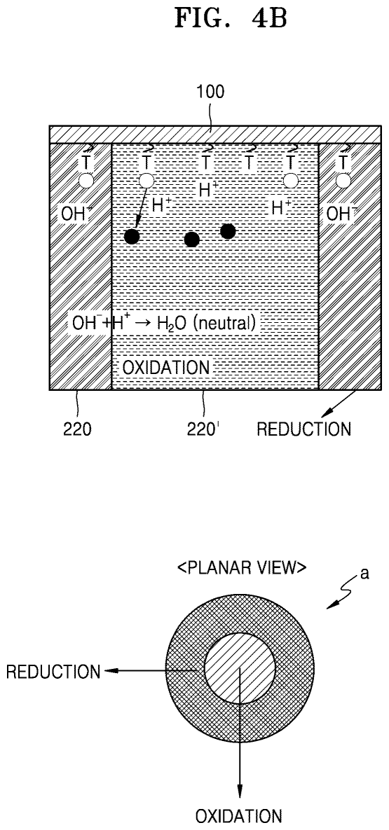

FIG. 4A is a diagram illustrating another embodiment of a nucleic acid synthesis apparatus, and electrode structures of a first substrate and a second substrate. In FIG. 4A, for convenience of explanation, a four-well array is displayed on the second substrate.

In FIG. 4A, the nucleic acid synthesis apparatus includes an electrode 110 on a first substrate 100, and an electrode 220 in a certain area of the second substrate 200 facing the electrode of the first substrate. The electrode of the first substrate may be a working electrode, and the electrode of the second substrate may be a reference electrode. The electrode of the first substrate may be a large-area electrode. The electrode of the first substrate may be a flat electrode. The electrode of the first substrate may be disposed on the nucleic acid synthesis area or the surface including it, for example, the entire surface of the first substrate.

The second substrate may include an array of a plurality of regions, each region may be in operable communication with a reference electrode, and each reference electrode may be in operable communication with to a switch and a controller. The region may be a region defined by the electrode of the first substrate and the reference electrode of the second substrate. The switch may be a CMOS. The controllers (V_{w1}, V_{R1}) may provide or may control electrical signals that instruct opening and closing of the switch. The controller may position-specifically activate electrodes at a specific position through the switch. For example, the controller may apply a voltage to generate acid to an electrode, to a first electrode, or to an array of first electrodes. The generated acid may cause a deprotection reaction to occur. The first substrate and the second substrate aligned may be incubated together or separately from the electrode activation.

In FIG. 4A, the nucleic acid synthesis apparatus includes a first substrate 100 without electrodes, and two electrodes 220, 220′ facing the nucleic acid synthesis area of the first substrate each in a predetermined area of the second substrate. The electrodes of the second substrate may be the reference electrode 220 and the working electrode 220′ (part A). The reference electrode and working electrode of the second substrate may be flat electrodes. The reference electrode and the working electrode of the second substrate may be arranged concentrically in a plane. Specifically, the reference electrode may be arranged concentrically in a plane with respect to the working electrode of the second substrate. The reference electrode and the working electrode may be disposed directly adjacent to each other or an insulating layer may be disposed therebetween.

The second substrate may include an array of a plurality of regions, and a reference electrode and a working electrode may be in operable communication with each region, and each reference electrode and working electrode may be in operable communication with a switch and a controller (part B). The region may be a flat region. The area may be defined by each the reference electrode and the working electrode. The switch may be a CMOS. The controllers (V_{w1}, V_{R1}) may provide or control electrical signals that instruct opening and closing of the switch. The controllers may position-specifically activate electrodes at a specific position through the switch, for example, a voltage may be applied to generate acid. The generated acid may cause a deprotection reaction to occur. The aligned first substrate and second substrate may be incubated together or separately from the electrode activation.

FIG. 4B is an enlarged view of the chemical reaction occurring in part A of FIG. 4A. As shown in FIG. 4B, when the reference electrode 220 of the second substrate is activated, OH− ions may be generated by a reduction reaction in the region between the nucleic acid synthesis region of the first substrate and the reference electrode 220 of the second substrate, and H⁺ ions may be generated by an oxidation reaction in the region between the nucleic acid synthesis region of the first substrate and the working electrode 220′ of the second substrate. Accordingly, the generated H⁺ ions may be isolated in the region between the nucleic acid synthesis region of the first substrate and the working electrode of the second substrate due to the generated OH-ions, so that the deprotection reaction or other reactions may be accurately and positionally controlled. A planar view “a” of the region between the nucleic acid synthesis region of the first substrate and the reference electrode 220 of the second substrate (labeled “reduction) and the region between the nucleic acid synthesis region of the first substrate and the working electrode 220′ of the second substrate is also shown in FIG. 4B.

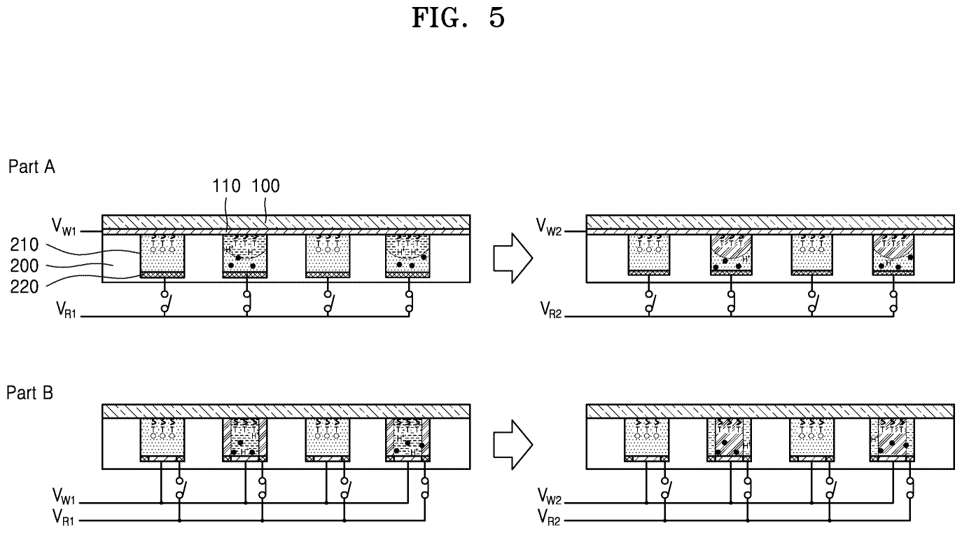

FIG. 5 is a diagram showing electrode structures of a first substrate and a second substrate, as another embodiment of a nucleic acid synthesis apparatus. In FIG. 5, for convenience of explanation, a four-well array is displayed on the second substrate. The nucleic acid synthesis apparatus of FIG. 5 is different from the nucleic acid synthesis apparatus of FIG. 3 in that a certain region of the first substrate is an array of wells.

In part A of FIG. 5, the nucleic acid synthesis apparatus includes an electrode 110 on a first substrate 100, and an electrode facing the electrode of the first substrate 220 in each well of the second substrate 200. The electrode of the first substrate may be a working electrode, and the electrodes of the second substrate may be reference electrodes. The electrode of the first substrate may be a large-area electrode. The electrode of the first substrate may be a flat electrode. The electrode of the first substrate may be disposed on the nucleic acid synthesis area or the surface including it, for example, the entire surface of the first substrate.

The second substrate includes an array of a plurality of wells 210, each well may be in operable communication with a reference electrode, and each reference electrode may be in operable communication with a switch and a controller. The well may be a microwell, nanowell, or picowell. The switch may be a CMOS. The controllers (V_{w1}, V_{R1}, or V_{w1}, V_{R2}) may provide or control an electrical signal instructing opening and closing of the switch. The controllers may position-specifically activate the electrodes of the wells at a specific position through the switch, for example, a voltage may be applied to generate acid. The generated acid may cause a deprotection reaction to occur. The aligned first substrate and second substrate may be incubated together or separately from the electrode activation.

In B of FIG. 5, the nucleic acid synthesis apparatus includes a first substrate not including electrodes, and includes two electrodes in each well of the second substrate facing the nucleic acid synthesis area of the first substrate.

The electrodes of the second substrate may be a reference electrode and a working electrode. The reference electrode and working electrode of the second substrate may be flat electrodes. The reference electrode and the working electrode of the second substrate are arranged concentrically in a plane. Specifically, the reference electrode is arranged concentrically in a plane with respect to the working electrode of the second substrate. The reference electrode and the working electrode may be disposed directly adjacent to each other or an insulating layer may be disposed therebetween.

The second substrate includes an array of a plurality of wells, each well is connected to a reference electrode and a working electrode, and each of the reference electrodes and the working electrode is functionally connected to a switch and a controller. The well may be a microwell, nanowell, or picowell. The switch may be CMOS. The controllers (V_{w1}, V_{R1}, or V_{w1}, V_{R2}) may provide or control an electrical signal instructing opening and closing of the switch. The controllers may position-specifically activate the electrodes of the wells at a specific position through the switch, for example, a voltage is applied to generate acid. The generated acid may cause a deprotection reaction to occur. The first substrate and the second substrate aligned may be incubated together with or separately from the electrode activation.

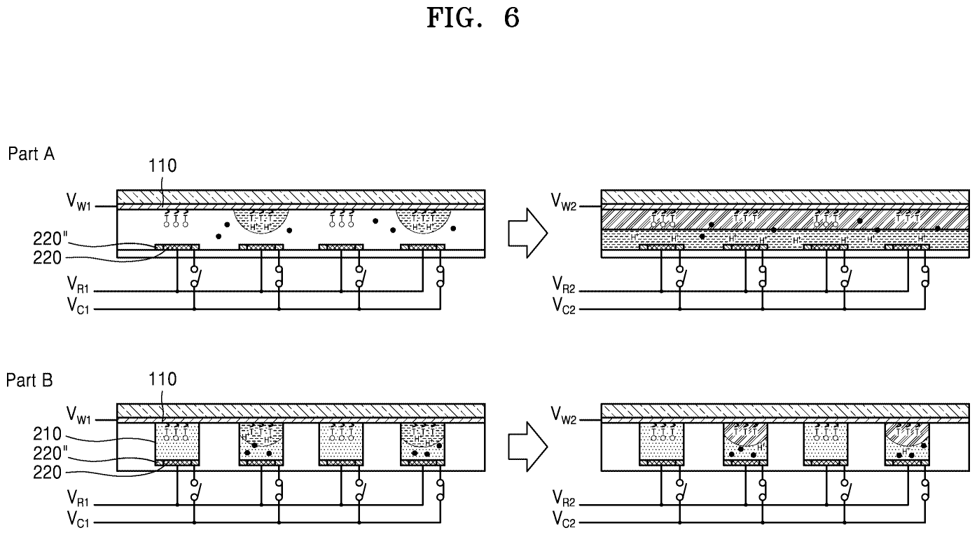

FIG. 6 is a diagram illustrating another embodiment of a nucleic acid synthesis apparatus, and electrode structures of a first substrate and a second substrate. In FIG. 6, for convenience of explanation, a four-well array is displayed on the second substrate. The nucleic acid synthesis apparatus of FIG. 6 includes three electrodes. Specifically, the nucleic acid synthesis apparatus includes one electrode on a first substrate and two electrodes on a second substrate.

In part A of FIG. 6, the nucleic acid synthesis apparatus includes an electrode 110 on a first substrate, and two electrodes 220, 220″ in each region of the second substrate facing the electrodes on the first substrate. The electrode 110 of the first substrate may be a working electrode, and the electrode of the second substrate may be a reference electrode 220 and a counter electrode 220″. The electrode of the first substrate may be a large-area electrode. The electrode of the first substrate may be a flat electrode. The electrode of the first substrate may be installed on the nucleic acid synthesis area or the surface including it, for example, the entire surface of the first substrate.

The second substrate may include an array of a plurality of regions, each region may be in operable communication with a reference electrode and a count electrode, and each of the reference electrode and the counter electrode may be in operable communication with a switch and a controller. The reference electrode and counter electrode of the second substrate may be flat electrodes. The reference electrode and counter electrode of the second substrate may be arranged concentrically in a plane. Specifically, the reference electrode may be arranged concentrically on a plane with respect to the counter electrode of the second substrate. The reference electrode and the counter electrode may be disposed directly adjacent to each other or an insulating layer may be disposed therebetween.

The region may be a micro, a nano, or a pico region. The switch may be a CMOS. The controllers (V_{w1}, V_{R1}, V_{C1}, or V_{W2}, V_{R2}, V_{C2}) may provide or control an electrical signal instructing opening and closing of the switch. The controllers may position-specifically activate electrodes at a specific position through the switch, for example, a voltage may be applied to generate acid (left side of part A of FIG. 6). The generated acid may cause a deprotection reaction to occur. The first substrate and the second substrate aligned may be incubated together or separately from the electrode activation. After acid generation or completion of incubation, the first substrate and the second substrate aligned may activate all working electrodes on the second substrate such that the nucleic acid synthesis region on the first substrate is dominated by OH-ions, and the reference electrode and counter electrode on the second substrate is dominated by H⁺ ions (right side of part A of FIG. 6). Accordingly, the unprotected end of the growing strand of the first substrate may be protected from H⁺ ions.

In part B of FIG. 6, the nucleic acid synthesis apparatus includes an electrode 110 on a first substrate, and includes two electrodes 220, 220″ facing the nucleic acid synthesis region of the first substrate in each well 210 of the second substrate.

The electrodes of the second substrate may be a reference electrode 220 and a counter electrode 220″. The reference electrode 220 and the counter electrode 220″ of the second substrate may be flat electrodes. The reference electrode and counter electrode of the second substrate may be arranged concentrically in a plane. Specifically, the reference electrode may be arranged concentrically on a plane with respect to the counter electrode of the second substrate. The reference electrode and the counter electrode may be disposed directly adjacent to each other or an insulating layer may be disposed therebetween.

The second substrate may include an array of a plurality of wells, each well may be in operable communication with a reference electrode and a counter electrode, and each reference electrode and counter electrode may be in operable communication with a switch and a controller. The well may be a microwell, a nanowell, or a picowell. The switch may be a CMOS. The controllers (V_{w1}, V_{R1}, V_{C1}, or V_{W2}, V_{R2}, V_{C2}) may provide or control an electrical signal instructing opening and closing of the switch. The controller may position-specifically activate the electrodes of the wells at a specific position through the switch, for example, a voltage may be applied to generate acid. The generated acid may cause a deprotection reaction to occur. The first substrate and the second substrate aligned may be incubated together with or separately from the electrode activation.

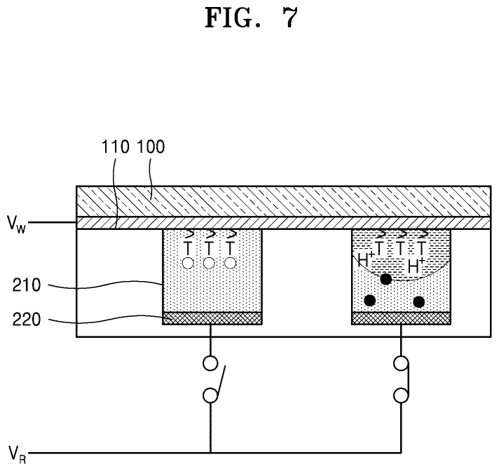

FIGS. 7 to 14 are diagrams illustrating embodiments of a nucleic acid synthesis apparatus, classified according to the presence or absence of electrodes in operable communication with the nucleic acid synthesis region of the first substrate, whether the number of electrodes in operable communication with two electrodes or three electrodes, and whether the region that may include the deprotection solution is a flat surface or a well.

FIGS. 7 to 10 are diagrams illustrating embodiments of a nucleic acid synthesis apparatus including an electrode in operable communication with the nucleic acid synthesis region of the first substrate.

Specifically, FIG. 7 is a diagram illustrating an embodiment of a nucleic acid synthesis apparatus including an electrode 110 in operable communication with the nucleic acid synthesis region of the first substrate 100, in which the region capable of including the deprotection solution has a shape of well 210, and one electrode 220 may be in operable communication with each well. As shown in FIG. 7, the electrode of the first substrate may be a working electrode, and the electrode of the second substrate may be a reference electrode.

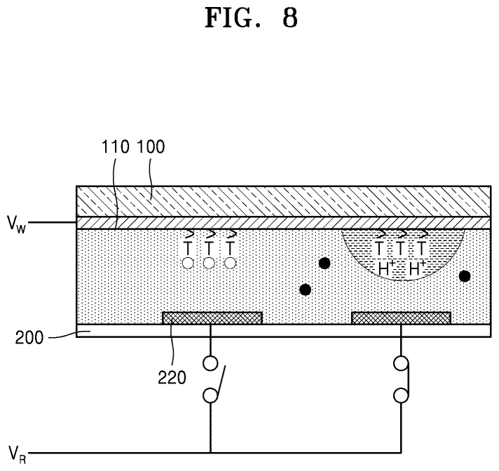

FIG. 8 is a diagram illustrating an embodiment of a nucleic acid synthesis apparatus including an electrode 110 in operable communication with the nucleic acid synthesis region of the first substrate 100, in which the second substrate 200 has a region that may include a deprotection solution in the form of a flat region, and one electrode 220 may be in operable communication with each region. As shown in FIG. 8, the electrode of the first substrate may be a working electrode, and the electrode of the second substrate may be a reference electrode.

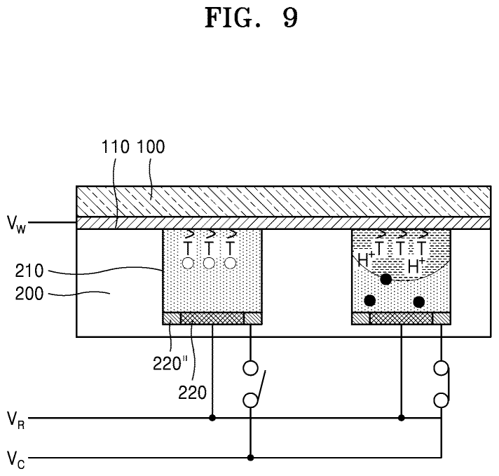

FIG. 9 is a diagram illustrating an embodiment of a nucleic acid synthesis apparatus including electrodes in operable communication with the nucleic acid synthesis region of the first substrate, in which the region capable of including the deprotection solution has the shape of a well 210, and two electrodes may be in operable communication with each well.

As shown in FIG. 9, the electrode 110 of the first substrate 100 may be a working electrode, and one of the electrodes of the second substrate 200 may be a reference electrode 220 and the other may be a counter electrode 220″. The reference electrode and counter electrode may be flat electrodes. The reference electrode and the counter electrode may be flat electrodes arranged in a concentric direction. With respect to the reference electrode, the counter electrode may be a flat electrode arranged in a concentric direction. The reference electrode and the counter electrode may be directly adjacent to each other (e.g., disposed directly adjacent and connected), or an insulating layer may be disposed therebetween.

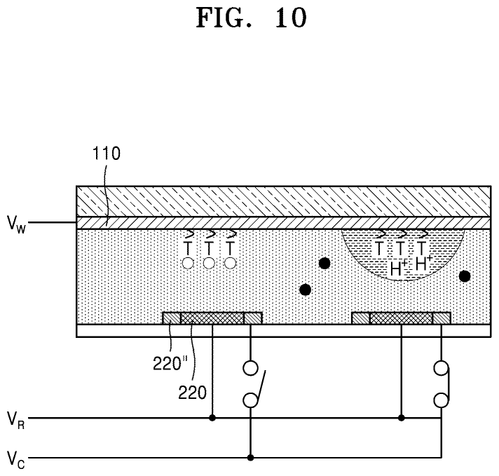

FIG. 10 is a diagram illustrating an embodiment of a nucleic acid synthesis apparatus including electrodes in operable communication with the nucleic acid synthesis region of the first substrate, in which the region capable of including the deprotection solution has a shape of flat region, and two electrodes may be in operable communication with each region.

As shown in FIG. 10, the electrode 110 of the first substrate may be a working electrode, and one of the electrodes of the second substrate may be a reference electrode 220 and the other may be a counter electrode 220″. The reference electrode and counter electrode may be flat electrodes. The reference electrode and the counter electrode may be flat electrodes arranged in a concentric direction. With respect to the reference electrode, the counter electrode may be a flat electrode arranged in a concentric direction. The reference electrode and the counter electrode may be disposed directly adjacent to each other (e.g., disposed directly adjacent and connected), or an insulating layer may be disposed therebetween.

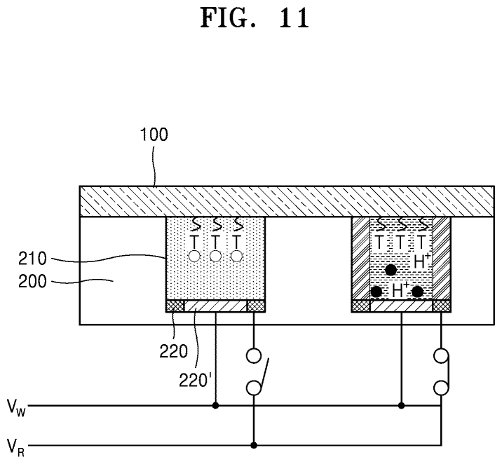

FIGS. 11 to 14 are diagrams illustrating embodiments of a nucleic acid synthesis apparatus not including an electrode in operable communication with the nucleic acid synthesis region of the first substrate.

Specifically, FIG. 11 is a diagram illustrating an embodiment of a nucleic acid synthesis apparatus not including an electrode in operable communication with the nucleic acid synthesis region of the first substrate 100, in which the region capable of including the deprotection solution has the shape of well 210, and two electrodes may be in operable communication with each well. As shown in FIG. 11, one of the electrodes of the second substrate 200 may be a working electrode 220′, and another may be a reference electrode 220. The reference electrode and working electrode may be flat electrodes. The reference electrode and the working electrode may be flat electrodes arranged in a concentric direction. With respect to the working electrode, the reference electrode may be a flat electrode arranged in a concentric direction. The reference electrode and the working electrode may be disposed directly adjacent to each other (e.g., disposed directly adjacent and connected), or an insulating layer may be disposed therebetween.

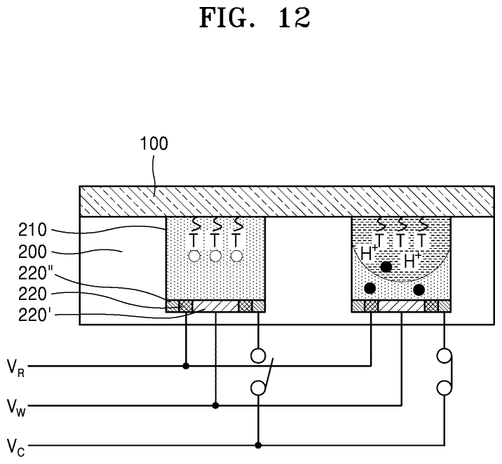

FIG. 12 is a diagram illustrating an embodiment of a nucleic acid synthesis apparatus not including an electrode in operable communication with the nucleic acid synthesis region of the first substrate 100, in which the region capable of including the deprotection solution has a shape of a well region 210, and three electrodes may be in operable communication with each region. As shown in FIG. 12, one of the electrodes of the second substrate 200 may be a working electrode 220′, another may be a reference electrode 220, and another may be a counter electrode 220″. The working electrode, reference electrode, and counter electrode may be flat electrodes. The working electrode, reference electrode, and counter electrode may be flat electrodes arranged in a concentric direction. With respect to the working electrode, the reference electrode and the counter electrode may be flat electrodes arranged in a concentric direction. With respect to the working electrode, the working electrode may be a flat electrode arranged concentrically in the order of the reference electrode and the counter electrode. The working electrode, reference electrode, and counter electrode may be disposed directly adjacent to each other (e.g., disposed directly adjacent and connected), or an insulating layer may be disposed therebetween.

One of the electrodes of the second substrate may be a working electrode and the other may be a reference electrode. The reference electrode and counter electrode may be flat electrodes. The reference electrode and the working electrode may be flat electrodes arranged in a concentric direction. With respect to the working electrode, the reference electrode may be a flat electrode arranged in a concentric direction. The reference electrode and the working electrode may be disposed directly adjacent to each other (e.g., disposed directly adjacent and connected), or an insulating layer may be disposed therebetween.

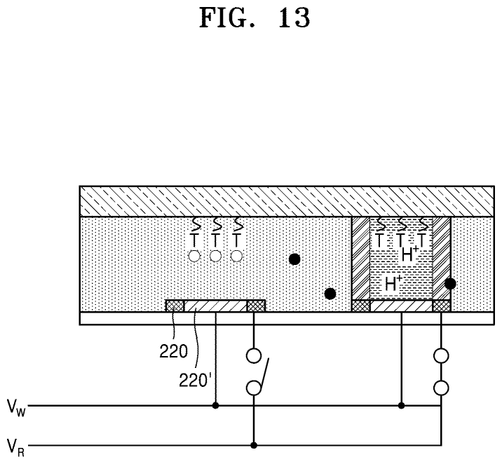

FIG. 13 is a diagram illustrating an embodiment of a nucleic acid synthesis apparatus not including an electrode in operable communication with the nucleic acid synthesis region of the first substrate, in which the region capable of including the deprotection solution has a flat shape, and two electrodes 220 and 220′ may be in operable communication with each region. As shown in FIG. 13, one of the electrodes of the second substrate may be a working electrode 220′, and the other may be the reference electrode 220. The working electrode and reference electrode may be flat electrodes. The working electrode and the reference electrode may be flat electrodes arranged in a concentric direction. With respect to the working electrode, the reference electrode may be a flat electrode arranged in a concentric direction. The working electrode and the reference electrode may be disposed directly adjacent to each other (e.g., disposed directly adjacent and connected), or an insulating layer may be disposed therebetween.

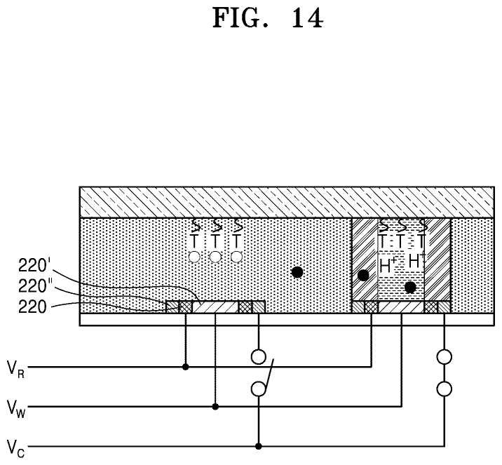

FIG. 14 is a diagram illustrating an embodiment of a nucleic acid synthesis apparatus not including an electrode in operable communication with the nucleic acid synthesis region of the first substrate, in which the region capable of including the deprotection solution has a shape of flat region, and three electrodes 220, 220′, 220″ may be in operable communication with each region. As shown in FIG. 14, one of the electrodes of the second substrate may be a working electrode 220′, another may be a reference electrode 220, and another may be a counter electrode 220″. The working electrode, reference electrode, and counter electrode may be flat electrodes. The working electrode, reference electrode, and counter electrode may be flat electrodes arranged in a concentric direction. With respect to the working electrode, the reference electrode and the counter electrode may be flat electrodes arranged in a concentric direction. The working electrode may be a flat electrode arranged concentrically in the order of the reference electrode and the counter electrode. The working electrode, reference electrode, and counter electrode may be disposed directly adjacent to each other (e.g., disposed directly adjacent and connected), or an insulating layer may be disposed therebetween.

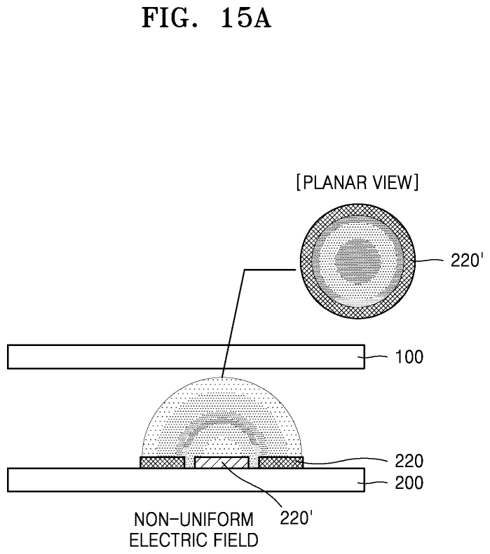

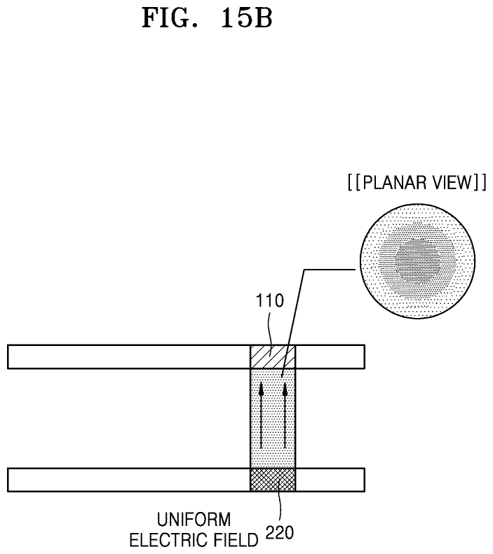

FIGS. 15A and 15B are a schematic diagrams illustrating an electric field in relation to the electrode structure of the nucleic acid synthesis apparatus according to an embodiment. In FIG. 15A, the nucleic acid synthesis apparatus does not include an electrode in operable communication with the nucleic acid synthesis region of the first substrate 100, two electrodes 220, 220′ may be disposed in a concentric direction to the second substrate, and one of the electrodes of the second substrate may be a working electrode 220′, and the other may be the reference electrode 220. The working electrode may be located toward the center of the concentric circle compared to the reference electrode, and an insulating layer may be disposed between the two electrodes to space them apart. In FIG. 15B, the nucleic acid synthesis apparatus includes an electrode 110 in operable communication with the nucleic acid synthesis region of the first substrate 100, and an electrode 220 facing the electrode of the first substrate may be in operable communication with the second substrate 200. The electrode of the first substrate may be a working electrode 110, and the electrode of the second substrate may be a reference electrode 220.

FIG. 15A shows that the highest electric field may be applied between the working electrode and the reference electrode, such that the electric field is weakly applied to the center of the working electrode, and the generated hydrogen ions diffuse laterally, so that the area of the applied electric field becomes wider and non-uniform. FIG. 15B shows when one electrode is present in the nucleic acid synthesis region of the first substrate and an electrode facing the electrode of the first substrate is present on the second substrate, the applied electric field may be uniform.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An apparatus for synthesizing nucleic acids, comprising:
a first substrate comprising a surface on which the nucleic acids are synthesized, the surface comprising a nucleic acid synthesis region functionalized to immobilize the nucleic acids or nucleic acid precursors;
a second substrate comprising a deprotection solution, wherein the second substrate comprises an array of regions capable of comprising the deprotection solution, an array of first electrodes in operable communication with the regions and configured to induce electrochemical acid generation in the deprotection solution, and a first electrode controller in operable communication with the array of the first electrodes and configured to apply a voltage to the first electrodes and to remove the voltage from the first electrodes;
a transporter configured to move the first substrate or the second substrate, wherein the transporter is in operable communication with the first substrate or the second substrate, and the transporter is configured to align the first substrate and the second substrate with each other; and
a transporter controller in operable communication with the transporter,
wherein the transporter controller is configured to
i) direct the transporter to move the first substrate in a direction toward or away from the second substrate, or
ii) direct the transporter to move the second substrate in a direction toward or away from the first substrate.

2. The apparatus of claim 1, wherein the first substrate comprises electrodes facing the first electrodes of the second substrate.

3. The apparatus of claim 1, wherein each electrode of the array of the first electrodes comprises two electrodes or three electrodes.

4. The apparatus of claim 3, wherein an insulating layer is included between each of the electrodes, wherein one of the electrodes is configured to generate H⁺ ions, and another of the electrodes is configured to generate OH− ions.

5. The apparatus of claim 1, wherein the nucleic acid precursors are protected nucleosides or single-stranded nucleic acids.

6. The apparatus of claim 1, wherein the regions capable of comprising the deprotection solution comprise an array of wells.

7. The apparatus of claim 6, wherein the array of the first electrodes is operably connected to each well of the array of wells.

8. The apparatus of claim 1, wherein the transporter comprises a driving means.

9. The apparatus of claim 1, further comprising an alignment sensor and an alignment controller,
wherein the alignment sensor is functionally connected between the first substrate and the second substrate, and is configured to measure horizontal plane misalignment between the first substrate and the second substrate,
the alignment controller is functionally coupled to the alignment sensor, is configured to receive horizontal plane misalignment from the alignment sensor, and is configured to compare the horizontal plane misalignment to a misalignment threshold,
when the horizontal plane misalignment is greater than the misalignment threshold, the alignment controller is configured to instruct the transporter controller to move the transporter in a direction in which the horizontal plane misalignment decreases, and
when the horizontal plane misalignment is less than or equal to the misalignment threshold, the alignment controller is configured to send an alignment acknowledgment to the transport controller.

10. The apparatus of claim 1, wherein the solution comprises a reversible redox pair.

11. The apparatus of claim 10, wherein the solution comprises hydroquinone and benzoquinone.

12. The apparatus of claim 1, further comprising a bulk reagent substrate comprising a bulk reagent.

13. The apparatus of claim 12, further comprising a second transporter connected to the first substrate or the bulk reagent substrate, the second transporter being configured to advance toward or retreat from the bulk reagent substrate or the first substrate.

14. The apparatus of claim 13, wherein the bulk reagent substrate comprises a bulk liquid reagent capable of contacting the entire surface of the first substrate on which the nucleic acids are synthesized.

15. The apparatus of claim 14, wherein the bulk liquid reagent is a coupling reagent, a capping reagent, or an oxidation reagent.

16. The apparatus of claim 12, wherein the bulk reagent substrate comprises a plurality of bulk reagent substrates each comprising a coupling reagent, a capping reagent, or an oxidation reagent.

17. A method for synthesizing nucleic acids, comprising:
identifying a target nucleic acid sequence to be synthesized;
providing a first substrate having a functionalized surface to immobilize the nucleic acids or nucleic acid precursors;
aligning the first substrate with a second substrate in operable communication with an array of first electrodes, wherein the second substrate comprises a deprotection solution;
applying a voltage to the array of first electrodes to induce acid generation for deprotection; and
aligning the first substrate with a first bulk reagent substrate, wherein the first bulk reagent substrate comprises a coupling reagent.

18. The method of claim 17, further comprising, after the aligning of the first substrate with the first bulk reagent substrate:
aligning the first substrate with a second bulk reagent substrate comprising a capping reagent;
aligning the first substrate with a third bulk reagent substrate comprising an oxidizing reagent; or
a combination thereof.

19. The method of claim 17, wherein the applying of a voltage to the array of first electrodes on the second substrate comprises address-specifically applying the voltage to a specific electrode of the array of first electrodes.

20. The method of claim 19, wherein the array of first electrodes is coupled to a first electrode controller, wherein the first electrode controller is a complementary metal-oxide-semiconductor field-effect transistor, an insulated-gate bipolar transistor, or a bipolar junction transistor.
